(19) United States
(12) Patent Application Publication
(10) Pub. No.: US 2013/0018424 A1
(43) Pub. Date: Jan. 17, 2013

(54) OSTEOTOMY AND ARTHRODESIS TREATMENT SYSTEM

(71) Applicant: Michael Subik, Atlantic Highlands, NJ (US)

(72) Inventor: Michael Subik, Atlantic Highlands, NJ (US)

(21) Appl. No.: 13/182,423

(22) Filed: Jul. 13, 2011

(51) Int. Cl. A61B 17/88; A61B 17/80

(52) U.S. Cl. 606/281; 606/286

(57) ABSTRACT

Methods and systems for applying anteroposterior compression to tarsal or carpal bones are provided. One such method includes securing a first fixation plate and a second fixation plate medial and lateral to the bones, respectively, with an anchoring bolt, wherein the anchoring bolt secures the first and second fixation plates relative to each other and the bones. The method may further include inserting a first and second pin into the bones through compression slots disposed on each of the first and second fixation plates. The method may further include using a first clamp to apply a first anteroposterior compression to the first and second pins associated with the first fixation plate and using a second clamp to apply a second anteroposterior compression to the first and second pins associated with the second fixation plate.

### FIELD

The subject technology generally relates to methods and systems for treating abnormally formed tarsal or carpal bones.

### BACKGROUND

Conventional methods for treating abnormally formed tarsal or carpal bones may include application of a fixation plate.

### SUMMARY

Various aspects of the subject technology provide methods and systems for creating a lateral compression and a first and second anteroposterior compression to abnormally formed tarsal or carpal bones (i.e. requiring osteotonmy or arthrodesis).

In various embodiments, methods and systems of the technology include the following: Item 1. A method for applying anteroposterior compression to tarsal or carpal bones, the method comprising: securing a first fixation plate and a second fixation plate medial and lateral to the bones, respectively, with an anchoring bolt, wherein the anchoring bolt secures the first and second fixation plates relative to each other and the bones; inserting a first pin into the bones through a first compression slot disposed on each of the first and second fixation plates; inserting a second pin into the bones through a second compression slot disposed on each of the first and second fixation plates; using a first clamp to apply a first anteroposterior compression to the first and second pins associated with the first fixation plate; using a second clamp to apply a second anteroposterior compression to the first and second pins associated with the second fixation plate; and maintaining the first and second anteroposterior compressions by securing the bones to the first and second fixation plates using a plurality of fixation screws.

Item 2. The method of Item 1, wherein magnitudes of the first and second anteroposterior compressions are different.

Item 3. The method of Item 2, wherein the first and second anteroposterior compressions are configured to create an abduction.

Item 4. The method of Item 2, wherein the first and second anteroposterior compressions are configured to create an adduction.

Item 5. The method of Item 1, wherein the first compression slot of each of the first and second fixation plates receives the first pin.

Item 6. The method of Item 1, wherein the second compression slot of each of the first and second fixation plates receives the second pin.

Item 7. The method of Item 1, wherein the second fixation plate receives a first pin different from the first pin of the first fixation plate.

Item 8. The method of Item 1, further comprising applying a medial to lateral compression between the first and second fixation plates using the first clamp.

Item 9. The method of Item 1, wherein the securing further comprises coupling the first and second fixation plates to the first clamp, wherein the first clamp has a plurality of engagement protrusions adapted to receive corresponding engagement openings disposed on the first and second fixation plates, wherein the plurality of engagement protrusions and corresponding engagement openings align the first and second fixation plates relative to each other.

Item 10. The method of Item 1, wherein the using a first clamp to apply a first anteroposterior compression to the first and second pins associated with the first fixation plate further comprises engaging the first and second pins with corresponding receptacles disposed on the clamping ends of the first clamp.

Item 11. The method of Item 1, wherein the first fixation plate is configured to receive a screw at varying axes while maintaining a substantially flush engagement between an outer surface of the plate and a screw head.

Item 12. The method of Item 1, wherein the first clamp is further configured with a drill bushing to facilitate drilling through the bones.

Item 13. A system for applying anteroposterior compression to tarsal or carpal bones, the system comprising: first and second fixation plates, wherein each plate has a respective anchoring hole configured to receive an anchoring bolt, wherein the anchoring bolt is configured to secure the first and second fixation plates relative to each other and the bones, and wherein the first and second fixation plates each have a first and second compression slot configured to receive a respective first and second pin; and first and second clamps, configured to apply a first and second anteroposterior compression, respectively, to the bones using the first and second pins after the pins have been inserted through the slots and the bones.

Item 14. The system of Item 13, wherein magnitudes of the first and second anteroposterior compressions are different.

Item 15. The system of Item 14, wherein the first and second anteroposterior compressions are configured to create an abduction.

Item 16. The system of Item 14, wherein the first and second anteroposterior compressions are configured to create an adduction.

Item 17. The system of Item 13, wherein the first and second fixation plates each have a plurality of openings for coupling the plates to the first clamp.

Item 18. The system of Item 13, wherein clamping ends of the first and second clamps have receptacles that are configured to engage the first and second pins.

Item 19. The system of Item 13, wherein the first fixation plate is configured to receive a screws at a varying axis while maintaining a substantially flush engagement between an outer surface of the plate and a screw head.

Item 20. The system of Item 13, wherein the first fixation plate has an amendable portion for contouring to the bones.

Item 21. The system of Item 13, wherein the first fixation plate has a cleat to engage the bones.

Item 22. The system of Item 13, wherein the first clamp has a drill bushing to facilitate drilling through the bones.

Item 23. The system of Item 13, further comprising the first and second pins configured to be inserted through the bones.

Item 24. The system of Item 13, further comprising the first and second pins configured to be inserted into the bones.

Item 25. The system of Item 24, wherein the first pin of the first compression slot of the first fixation plate is different from the first pin of the first compression slot of the second fixation plate.

Item 26. The system of Item 13, further comprising a variable axis screw configured to be inserted into the bones.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 illustrates a system for creating a lateral compression and a first and second anteroposterior compression, in accordance with various aspects of the subject technology.

FIG. 2 illustrates a clamp, in accordance with various aspects of the subject technology.

FIGS. 3A-3E illustrate various views of a clamping end of a clamp, in accordance with various aspects of the subject technology.

FIGS. 4A-4B illustrate a front and side view of a fixation plate, in accordance with various aspects of the subject technology.

FIGS. 5A-5B illustrate a side and front view of a drill bushing, in accordance with various aspects of the subject technology.

FIGS. 6A-6B illustrate a side and front view of a collar, in accordance with various aspects of the subject technology.

FIG. 7 illustrates a system for creating a lateral compression, in accordance with various aspects of the subject technology.

FIG. 8 illustrates a cross section of a lateral compression system, in accordance with various aspects of the subject technology.

FIG. 9 illustrates a system for creating a first and second anteroposterior compression, in accordance with various aspects of the subject technology.

FIG. 10 illustrates a detailed view of a locking lobe of a fixation plate, in accordance with various aspects of the subject technology.

FIGS. 11A-11C illustrate fixation plates, in accordance with various aspects of the subject technology.

FIGS. 12A-12B illustrate a side and front view of a fixation plate, in accordance with various aspects of the subject technology.

FIGS. 13A-13B illustrate a nut, in accordance with various aspects of the subject technology.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology. Like components are labeled with identical element numbers for ease of understanding.

Conventional methods for treating abnormally formed tarsal or carpal bones (i.e., requiring osteotomy or arthrodesis) may include application of a fixation plate. The fixation plate may stabilize and compress the tarsal or carpal bones only in the medial-lateral direction and therefore, be incapable of compressing the tarsal or carpal bones in a longitudinal direction to compensate for abductory and adductory deformities. In addition, conventional fixation plates may not be configured to compress the tarsal or carpal bones in a medial-lateral direction to correct frontal plane deformity. Additionally, conventional fixation plates may not be configured to perform triplane or multiaxial correction without the assistance of an external fixation device or system. Moreover, conventional fixation plates may not be configured to interconnect with one another, thereby providing insufficient stability and/or torsional support.

Various aspects of the subject technology provide methods and systems for creating a lateral compression and a first and second anteroposterior compression to abnormally formed tarsal or carpal bones (i.e., requiring osteotomy or arthrodesis). The anteroposterior compression created by the subject technology, may correct abductory or adductory deformities in the tarsal or carpal bones. In addition, the subject technology may be configured to perform triplane or multiaxial correction without the assistance of an external fixation device or system.

In one aspect, the subject technology may be configured to interconnect fixation plates, thereby providing enhanced stability and torsional support, without an external fixation device. In addition, the interconnected fixation plates and/or the anteroposterior compression may facilitate the correction of frontal plane deformity. By creating the lateral and anteroposterior compression, the subject technology may facilitate rapid bone incorporation and healing, thereby promoting early weight-bearing of the bones.

In some aspects, the system may comprise a first and second fixation plate, wherein each plate is configured with at least one anchoring hole for securing the first and second fixation plates to the bones with an anchoring bolt and at least two compression slots for receiving pins and cannulated screws. The system may further comprise a first and second clamp, wherein the first and second clamps are configured to apply the first and second anteroposterior compressions, respectively, to the bones using the pins.

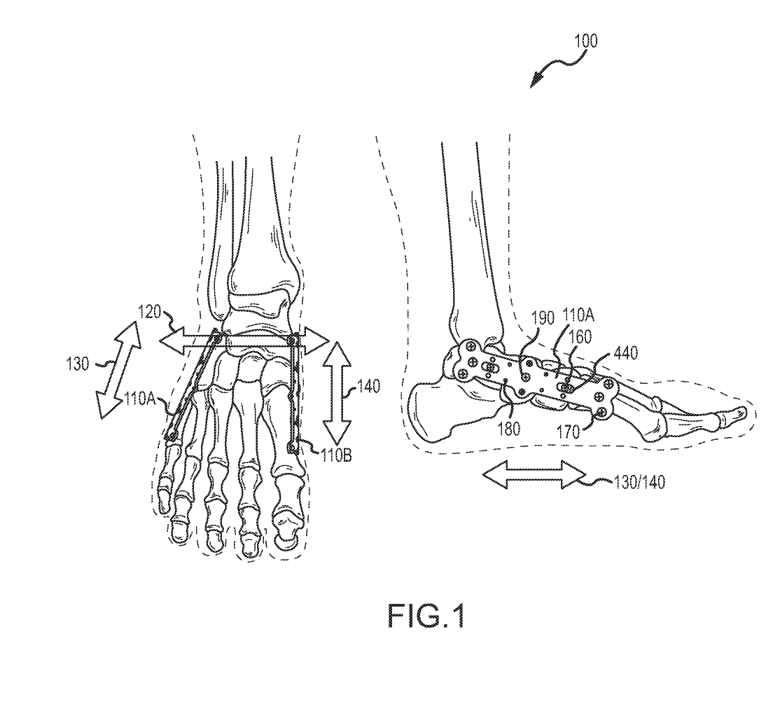

FIG. 1 illustrates a system 100 for creating a lateral compression 120 and a first and second anteroposterior compression, 130 and 140 respectively, in accordance with various aspects of the subject technology. In some aspects, a first and second fixation plate, 110A and 110B respectively, are disposed on the medial and lateral sides of the bones, respectively, and may create a lateral compression 120 on the bones with an anchoring bolt 190, as further described below. In other aspects, the first fixation plate 110A may create the first anteroposterior compression 130 with cannulated screws 160 and compression slots 440, as further described below. In other aspects, the second fixation plate 110B may create the second anteroposterior compression 140 with cannulated screws 160 compression 140 with cannulated screws 160 and compression slots 440, as further described below.

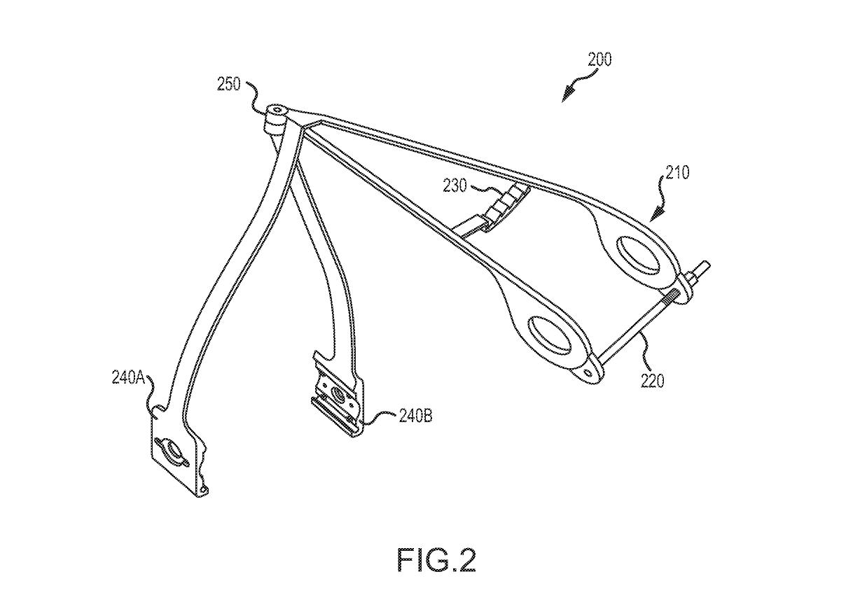

FIG. 2 illustrates a clamp 200, in accordance with various aspects of the subject technology. In some aspects, the clamp 200 may be configured for hand operation, by for example, utilizing finger and thumb openings 210 for facilitating operation of the clamp 200 by hand. In another aspect, the clamp 200 may comprise a clamping adjuster 220 for adjusting the clamping force of the clamp 200. For example, the clamping adjuster 220 may comprise a threaded screw and threaded nut engagement, whereby adjustment of the clamping force is accomplished by tightening or loosening of the threaded screw and nut. In one aspect, the clamp 200 may also comprise a position lock 230 for maintaining the position of a first clamping end 240A of the clamp 200 relative to a second clamping end 240B of the clamp 200. In another aspect of the subject technology, the clamp 200 may comprise a pivot 250 for translating motion on a first plane, to motion on a second plane perpendicular to the first plane.

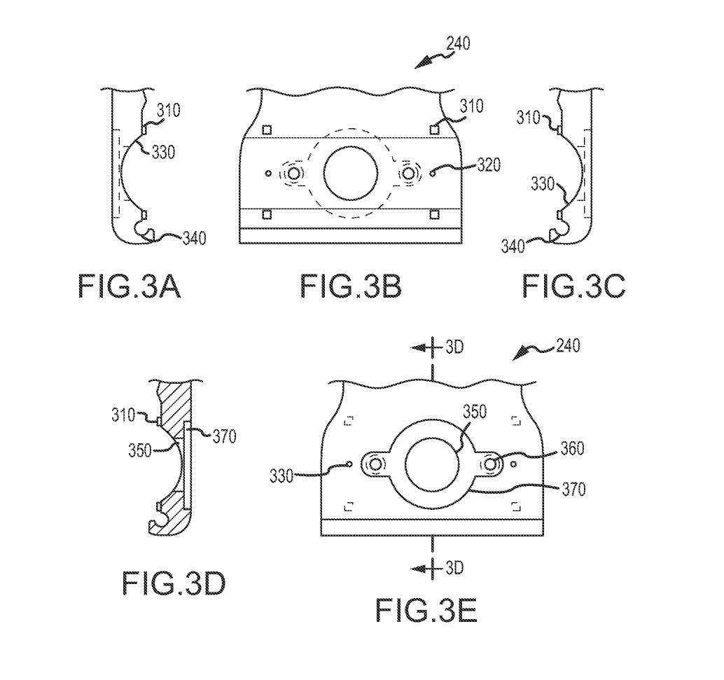

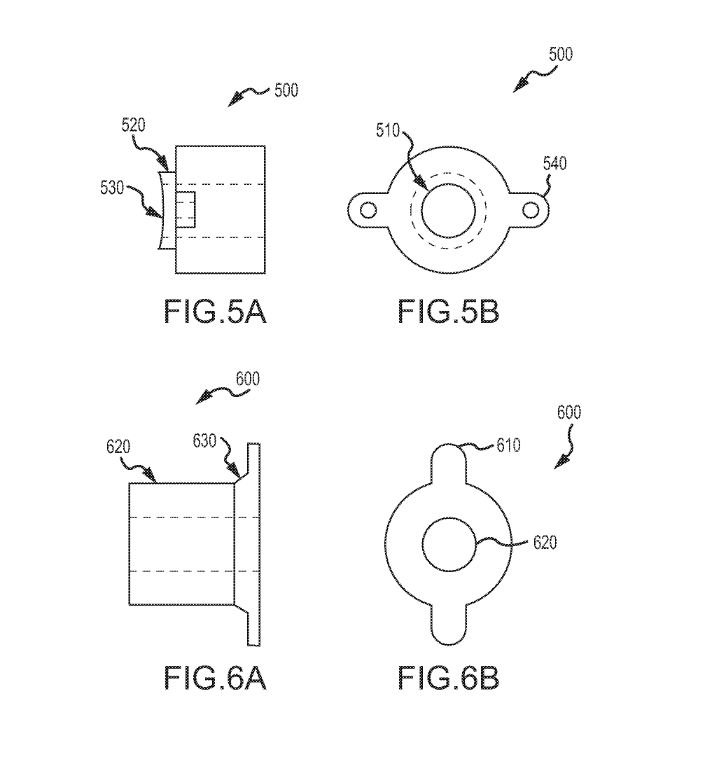

FIGS. 3A-3E illustrate various views of a clamping end 240 of a clamp 200, in accordance with various aspects of the subject technology. In some aspects, the clamping end 240 may comprise a plurality of engagement protrusions 310 adapted to receive corresponding engagement openings disposed on the fixation plates 110 and to couple and securely hold the fixation plates 110 onto the clamp 200, as further discussed below. In some aspects, the clamping end 240 may be configured with a plurality of openings 320 to facilitate the use of amendable wire for securing the fixation plates 110 to the clamp 200. For example, the plurality of openings 320 may be configured for use of K-wire to secure the fixation plates 110 to the clamping end 240 of the clamp 200. In one aspect, the clamping end 240 may be configured with a contour 330 for accommodating a cross-sectional profile of the fixation plates 110, as further described below. For example, for a fixation plate 110 with a rounded profile, the contour 330 may be configured with a rounded contour with sufficient clearance to prevent an interference between the clamping end 240 and the fixation plate 110. In some aspects, the clamping end 240 may be configured with a receptacle 340 for engaging a pin 900 and creating an anteroposterior compression, as further discussed below. In one aspect of the subject technology, the clamping end 240 may be configured to receive a drill bushing 500 for facilitating drilling through the bones (as shown in FIGS. 5A-5B). For example, the clamping end 240 may comprise an access hole 350 with a diameter appropriately sized to facilitate installation of hardware into the bones and onto the fixation plates 110, as further discussed below. In one aspect, the access hole may comprise a counterbore 370 configured to stabilize the drill bushing 500 onto the clamping end 240. In some aspects, the clamping end 240 may also comprise a plurality of threaded holes 360 configured to securely hold the drill bushing 500 onto the clamping end 240.

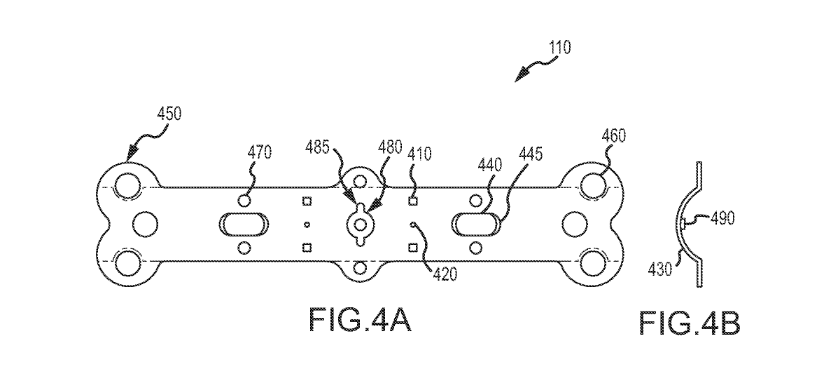

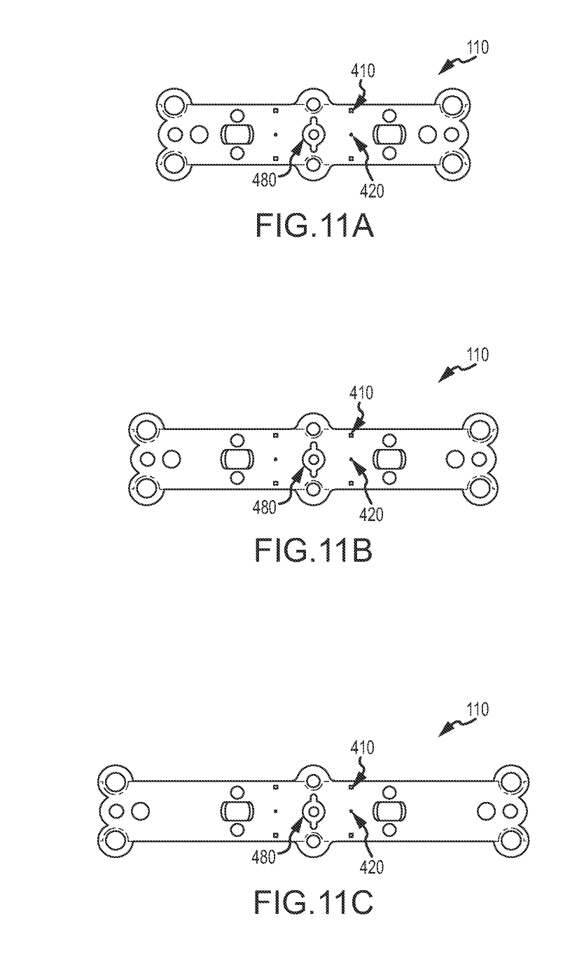

FIGS. 4A-4B illustrate a front and side view of the fixation plate 110, in accordance with various aspects of the subject technology. In one aspect of the subject technology, the fixation plate 110 may be manufactured from a suitable material, such as a metal or metal alloy with a sufficient rigidity and a minimal weight to adequately treat abnormally formed tarsal or carpal bones (i.e., requiring osteotomy or arthrodesis). For example, the fixation plate 110 may by manufactured from titanium. In some aspects, a first and second fixation plate, 100A and 100B respectively, are identical. In other aspects, the fixation plate 110 may have varying lengths. For example, referring to FIGS. 11A-11C, the fixation plate 110 may have a length of about 6 cm, 8 cm, 10 cm, or 12 cm.

Referring to FIGS. 4A-4B, in some aspects, the fixation plate 110 may comprise a plurality of amendable portions 450 to enable contouring of the fixation plate 110 to the outer profile shape and/or surface of the bones. For example, the plurality of amendable portions 450 may comprise tabs extending outward from the center of the fixation plate 110. In one aspect, to facilitate contouring of the fixation plate 110, the amendable portions 450 may comprise a neck for bending along the neck. In another example, the plurality of amendable portions 450 may have a thinner cross section than the non-amendable portions of the fixation plate 110 to facilitate bending toward the bones. In this example, the fixation plate 110 may have a general thickness of about 3 mm and the plurality of amendable portions 450 may have a general thickness of about 2 mm. In some aspects, the amendable portions 450 may have a height of about 2.5 cm. In other aspects, the fixation plate 110, without the amendable portions 450, may have a height of about 1.6 cm to 2 cm. In another aspect, an amendable portion 450 may comprise a hinge (not shown) to facilitate contouring of the amendable portion to the bones.

In one aspect, the fixation plate 110 may comprise a contour cross section 430 to facilitate a flush fit against the bones. For example, the contour 430 may comprise one-third of a tubular profile. In some aspects, to increase the torsional stiffness and/or stability of the fixation plate 110, the fixation plate may have a “T” or “I” beam cross section.

In some aspects, the fixation plate 110 may comprise a plurality of engagement openings 410 configured to receive the plurality of engagement protrusions 310 of the clamp 200 to couple and securely hold the fixation plate 110 onto the clamping end 240, as discussed above. In other aspects, the plurality of engagement openings 410 and the plurality of engagement protrusions 310 of the clamping end 240 align a first fixation plate 110A relative to a second fixation plate 110B. The plurality of engagement openings 410 may, for example, comprise square openings configured to receive square protrusions 310. In one aspect, the fixation plate 110 may comprise a plurality of openings 420 that are configured to accept a wire for holding the fixation plate 110 to the clamping end 240, by for example, the use of K-wire, as discussed above.

In some aspects, the fixation plate 110 may comprise a plurality of compression slots 440 for receiving pins 900 and cannulated screws 160, as further discussed below. In one aspect, the compression slot 440 may be configured to receive about a 2 mm diameter pin and/or 4 mm or 5 mm diameter cannulated screw 160. In some aspects the compression slot 440 may have a length of about 1 cm or more. In one aspect of the subject technology, the compression slot 440 may comprise a compression ramp 445 to facilitate an additional compression as the cannulated screw 160 is threaded into the bones. For example, the compression ramp 445 may comprise a beveled surface disposed at the ends of the slot, configured to engage a head of the cannulated screw 160 and thereby cause movement of the bones relative to the fixation plate 110. In this example, adjustment of the cannulated screw 160 may cause the head of the cannulated screw 160 to travel along the beveled surface, thereby moving the bones toward the center of the fixation plate 110 and thus causing an anteroposterior compression.

In some aspects, the fixation plate 110 may comprise a plurality of locking lobes 460 disposed along the amendable portions 450 and/or the fixation plate 110. Referring to FIG. 10, a detailed view of a locking lobe 460 is illustrated, in accordance with various aspects of the subject technology. The locking lobe 460 may comprise four upper primary lobes 1010 and four lower secondary lobes 1020 which are configured to received a variable axis screw 170, whereby the variable axis screw 170 securely attaches the amendable portions 450 and/or the fixation plate 110 to the bones. In some aspects, the locking lobe 460 is configured to accommodate up to a 10 degree variable axis for the variable axis screw 170. In some aspects, the locking lobe 460 may be configured to receive a 3.5 mm or 4.0 mm diameter variable axis screw 170. In one aspect, the fixation plate 110 may be configured so that a screw may be inserted into the bones at varying axes while maintaining a substantially flush engagement between an outer surface of the fixation plate 110 and a head of the screw. For example, the fixation plate 110 may be configured with a counterbore, countersink, or lobes (as described above) which enable the head of the screw to lay substantially flush with the outer surface of the fixation plate 110.

Referring to FIGS. 4A-4B, in some aspects, the fixation plate 110 may comprise a plurality of fixation holes 470 for receiving fixation screws 180, whereby the fixation screws 180 securely attach the fixation plate 110 to the bones. In some aspects, the plurality of fixation holes 470 are configured to receive a 3.5 mm, 4.0 mm, and/or 5.0 mm diameter fixation screw 180.

In some aspects, the fixation plate 110 may comprise an anchoring hole 480 configured to receive the anchoring bolt 190, whereby the anchoring bolt 190 creates a lateral compression between a first fixation plate 110A and a second fixation plate 110B, as described below. In some aspects, the anchoring hole 480 may be configured to receive a 5.0 mm diameter anchoring bolt 190. In one aspect, the anchoring hole 480 may be configured so that a head of the anchoring bolt 190 is flush with the surface of the fixation plate 110. For example, the anchoring hole 480 may comprise a countersink configured to receive the head of the anchoring bolt 190 and sized so that the head of the anchoring bolt 190 is flush with the surface of the fixation plate 110 once installed. In some aspects, the fixation plate may comprise a cleat 490 disposed on a surface of the fixation plate 110 facing the bones for facilitating placement and engagement of the fixation plate 110 onto the bones by preventing undesired movement of the fixation plate 110 relative to the bones during installation of the fixation plate 110 onto the bones. For example, a physician may remove material from the bones, thereby creating a notch, to accommodate the cleat 490 and subsequently position the fixation plate 110 onto the bones, ensuring that the cleat 490 is positioned in the notch. In some aspects the cleat 490 may be disposed proximal to the anchoring hole 480.

In some aspects, the anchoring hole 480 may be configured to receive a collar 600 and prevent the collar 600 from rotating in place, as further described below. For example, the anchoring hole 480 may comprise a plurality of cutouts 485 configured to receive corresponding tabs 610, as shown in FIGS. 6A and 6B. By positioning the tabs 610 of the collar 600 in the cutouts 485, the collar is prevented from rotating in place.

Referring to FIGS. 11A-11C, in some aspects, varying lengths of the fixation plate 110 may be configured to be securely held by the clamp 200. For example, by maintaining an identical physical arrangement between the engagement openings 410, the plurality of openings 420, and the anchoring hole 480, for varying lengths of the fixation plate 110, the clamp 200 may be utilized to couple and securely hold fixation plates 110 of different lengths.

FIGS. 5A-5B illustrate a side and front view of a drill bushing 500, in accordance with various aspects of the subject technology. In some aspects, the drill bushing 500 may comprise an inner diameter 510 configured to guide a drill bit through the bones. In other aspects, the drill bushing 500 may be temporarily positioned onto the clamp 200. For example, the drill bushing 500 may comprise a seat 520 configured to engage the counterbore 370 of the clamping end 240 of the clamp 200 (as shown in FIGS. 3A-3E). In other aspects, the drill bushing 500 may be secured to the clamp 200. For example, the drill bushing 500 may comprise a plurality of mounting tabs 540 configured to engage the threaded holes 360 of the clamping end 240 of the clamp 200 (as shown in FIGS. 3A-3E). In one aspect, the drill bushing 500 may comprise a contoured surface 530 configured to accept the contour cross section 430 of the fixation plate 110 without interference (as shown in FIGS. 4A-4B).

FIGS. 6A-6B illustrate a side and front view of the collar 600, in accordance with various aspects of the subject technology. In some aspects, the collar 600 may be configured to receive and secure the anchoring bolt 190, as further described below. For example, the collar 600 may comprise a threaded portion 620 adapted to engage a corresponding threaded portion of the anchoring bolt 190. In another aspect, the collar 600 may be adapted to use the anchoring hole 480 of the fixation plate 110 without modifying the fixation plate 110. For example, the collar 600 may comprise a chamfer 630 configured to accept the countersink of the anchoring hole 480 (as shown in FIGS. 4A-4B). In one aspect, the collar 600, once installed, may be flush with the surface of the fixation plate 110. As discussed above, the collar 600 may be prevented from rotating in the anchoring hole 480 by the tabs 610 and the cutouts 485 of the fixation plate 110 (as shown in FIGS. 4A-4B).

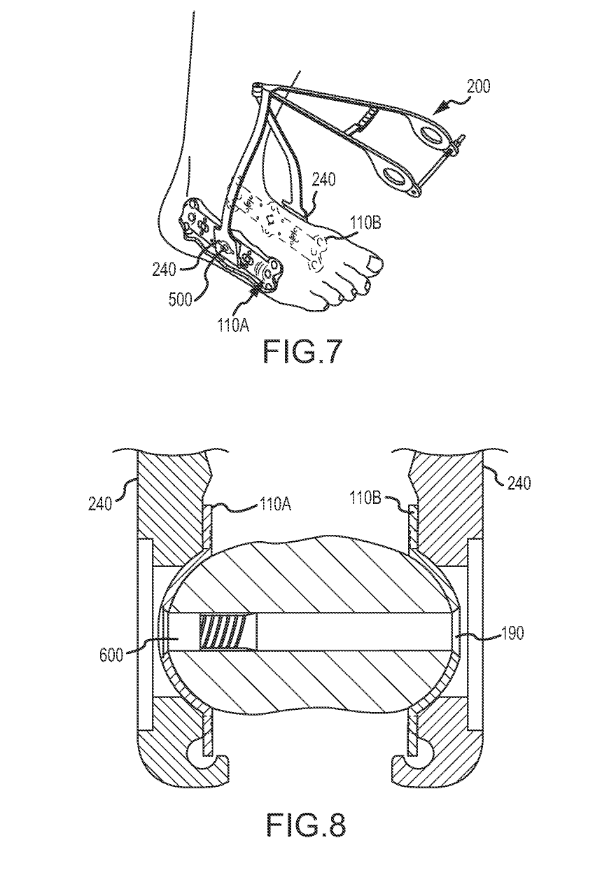

FIG. 7 illustrates a system 100 for creating a lateral compression 120, in accordance with various aspects of the subject technology. In one aspect of the subject technology, after the physician prepares a foot of a patient undergoing an osteotomy, material from the bone is removed to create the notch, as described above, to accommodate the cleat 490 of the first and second fixation plates, 110A and 110B respectively. Thereafter, the first and second fixation plates, 110A and 110B respectively, may be positioned onto the clamping ends 240 of the clamp 200 by utilizing the engagement protrusions 310 of the clamping ends 240 and the engagement openings 410 of the fixation plates, as described above. In one aspect, the clamping ends 240 align the first and second fixation plates, 110A and 110B respectively, so that the anchoring holes 480 of the first and second fixation plates, 110A and 110B respectively, are concentric. In some aspects, the engagement protrusions 310 of the clamping ends 240 and the engagement openings 410 of the fixation plates enables the first and second fixation plates, 110A and 110B respectively, to be manipulated as desired and necessary so that the fixation plates are disposed over the medial and lateral sides of the foot. In one aspect, the position of the first and second fixation plates, 11A and 110B respectively, relative to the medial and lateral sides of the foot may be maintained by engaging the position lock 230 of the clamp 200, as described above. In another aspect, if additional adjustment, force, or compression is necessary, then the clamping adjuster 220 of the clamp 200 may be utilized as desired, as described above.

In some aspects, after the first and second fixation plates, 110A and 110B respectively, are preliminarily positioned over the bones, the bones may be drilled to secure the first and second fixation plates, 110A and 110B respectively, to the bones and create, maintain, and/or apply the lateral compression 120. In some aspects, to aid in drilling the bones, the drill bushing 500 may be attached to the clamping ends 240. As illustrated in FIG. 7, the drill bushing 500 is attached to the clamping end 240 corresponding to the first fixation plate 110A. In this example, the drill bushing 500 is configured to guide a drill bit through the bones from the lateral side of the foot to the medial side of the foot, such that the drill bit creates a bore in the bones that is in alignment with the anchoring hole 480 of the second fixation plate 110B. In some aspects, the drill bit may comprise a radioluscent drill bit to aid in guiding the drill bit through the bones.

In some aspects, after removing the drill bushing 500 from the clamping end 240, the anchoring bolt 190 may be inserted into the anchoring hole 480 to threadedly engage the collar 600, thereby creating, maintaining, and/or applying the lateral compression 120. Referring to FIG. 8, a cross section of the bones, the clamping ends 240, the first and second fixation plates, 110A and 110B respectively, the anchoring bolt 190, and the collar 600 are illustrated. In this example, the anchoring bolt 190 is inserted into the anchoring hole 480 of the second fixation plate 110B and the collar 600 is inserted into the anchoring hole 480 of the first fixation plate 110A. In some aspects, the anchoring bolt 190 may comprise a solid core screw for strength and rigidity. As the anchoring bolt 190 engages the collar 600, the first and second fixation plates, 110A and 110B respectively, engage one another and laterally compress the bones in between the fixation plates.

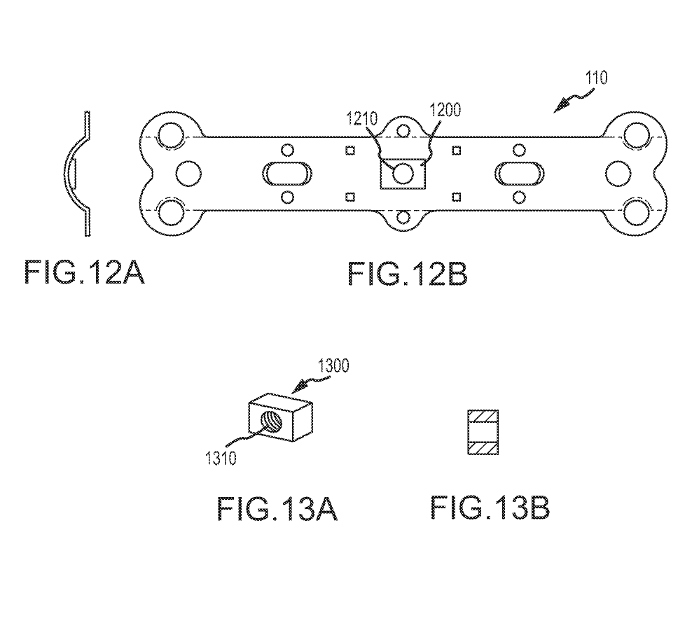

In another aspect, the fixation plates 110 may be configured with a socket 1200 configured to receive a nut 1300, as shown in FIGS. 12A-12B and 13A-13B. In some aspects, the socket 1200 may be disposed proximal to the anchoring hole 480, with a clearance hole 1210 concentric with the anchoring hole 480. In certain aspects, the socket 1200 may be configured to receive and securely hold the nut 1300 so that the threaded portion of the anchoring bolt 190 engages a threaded portion 1310 of the nut 1300. In this example, the anchoring bolt 190 may be inserted into the anchoring hole 480 of a first fixation plate and the nut 1300 may be inserted into the socket 1200 of a second fixation plate. As the anchoring bolt 190 engages the threaded portion 1310 of the nut 1300, the first and second fixation plates engage one another and laterally compress the bones in between the fixation plates.

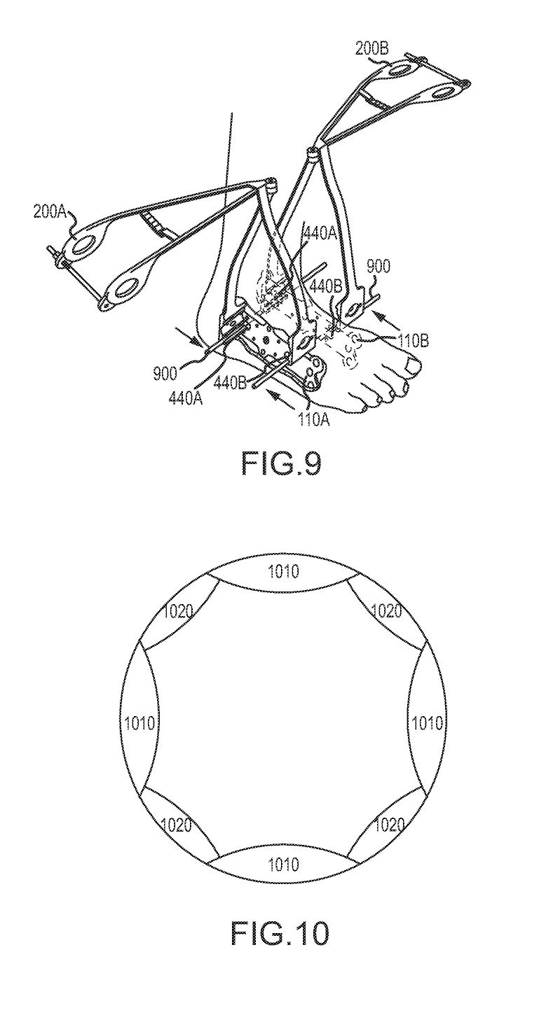

FIG. 9 illustrates a system 100 for creating a first and second anteroposterior compression, 130 and 140 respectively, in accordance with various aspects of the subject technology. In some aspects, after the first and second fixation plates, 110A and 110B respectively, are mechanically attached to each other with the anchoring bolt 190 and the collar 600, the clamp 200 may be removed from the first and second fixation plates, 110A and 110B respectively. In some aspects, to create the first and second anteroposterior compressions, 130 and 140 respectively, the bones may be drilled, using the fixation plate 110 as a guide, to create at least two bores through the bones. For example, the first bore may be drilled at the first compression slot 440A of the first fixation plate 11A and extend through to the corresponding first compression slot 440A of the second fixation plate 110B. The second bore may be drilled at the second compression slot 440B of the first fixation plate 110A, and extend through to the corresponding slot 440B of the second fixation plate 110B. A first and second pin 900 may then be inserted through the first and second bore. In some aspects, the pins 900 may comprise a 2 mm diameter guide wire.

In some aspects, a first clamp 200A engages the pins 900 via the receptacle 340 of the clamping ends 240, as described above. In one aspect, the first anteroposterior compression 130 may be created by actuating the first clamp 200A so that the pins 900 are moved toward the center of the first fixation plate 110A. In other aspects, to maintain the first anteroposterior compression 130, the fixation screws 180 may be installed at the fixation holes 470 of the first fixation plate 110A, as further described above. In various aspects, to maintain the first anteroposterior compression 130, the variable axis screws 170 may be installed at the locking lobes 460 disposed along the amendable portions 450 and/or the first fixation plate 110A, as further described above. In one aspect, the amendable portions 450 may be manipulated so that they lay flush against the bones, as described above.

In some aspects, a second clamp 200B engages the pins 900 via the receptacle 340 of the clamping ends 240, as described above. In certain aspects, the first and second clamps, 200A and 200B respectively, are identical. In one aspect, the second anteroposterior compression 140 may be created by actuating the second clamp 200B so that the pins 900 are moved toward the center of the second fixation plate 110B. In other aspects, to maintain the second anteroposterior compression 140, the fixation screws 180 may be installed at the fixation holes 470 of the second fixation plate 110B, as further described above. In various aspects, to maintain the second anteroposterior compression 140, the variable axis screws 170 may be installed at the locking lobes 460 disposed along the amendable portions 450 and/or the second fixation plate 110B, as further described above. In one aspect, the amendable portions 450 may be manipulated so that they lay flush against the bones, as described above. In some aspects, the first and second anteroposterior compressions, 130 and 140 respectively, may have different magnitudes with respect to each other. In one aspect, the first and second anteroposterior compressions, 130 and 140 respectively, may be configured to create an abduction. In another aspect, the first and second anteroposterior compressions, 130 and 140 respectively, may be configured to create an adduction.

In some aspects, the first compression slot 440A of the first fixation plate 110A may receive a first pin, inserted into the bones, while the first compression slot 440A of the second fixation plate 110B may receive a second pin, inserted into the bones. In some aspects, the second compression slot 440B of the first fixation plate 110A may receive a third pin, inserted into the bones, while the second compression slot 440B of the second fixation plate 110B may receive a fourth pin, inserted into the bones. In this example, the first clamp may engage the first and third pins of the first fixation plate 110A while the second clamp engages the second and fourth pins of the second fixation plates 110B.

In one aspect, after the first and second anteroposterior compressions, 130 and 140 respectively, are maintained, the first and second clamps, 200A and 200B respectively, may be removed. In one aspect, the cannulated screws 160 may be installed onto the bones using the pins 900 as a guide. In another aspect, as the cannulated screws 160 are driven into the bones, the compression ramp 445 of the compression slot 440 creates an additional anteroposterior compression as the head of the cannulated screw 160 engages the compression ramp 445, as further described above.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A method for applying anteroposterior compression to tarsal or carpal bones, the method comprising:
securing a first fixation plate and a second fixation plate medial and lateral to the bones, respectively, with an anchoring bolt, wherein the anchoring bolt secures the first and second fixation plates relative to each other and the bones;
inserting a first pin into the bones through a first compression slot disposed on each of the first and second fixation plates;
inserting a second pin into the bones through a second compression slot disposed on each of the first and second fixation plates;
using a first clamp to apply a first anteroposterior compression to the first and second pins associated with the first fixation plate;
using a second clamp to apply a second anteroposterior compression to the first and second pins associated with the second fixation plate; and
maintaining the first and second anteroposterior compressions by securing the bones to the first and second fixation plates using a plurality of fixation screws.

2. The method of claim 1, wherein magnitudes of the first and second anteroposterior compressions are different.

3. The method of claim 2, wherein the first and second anteroposterior compressions are configured to create an abduction.

4. The method of claim 2, wherein the first and second anteroposterior compressions are configured to create an adduction.

5. The method of claim 1, wherein the first compression slot of each of the first and second fixation plates receives the first pin.

6. The method of claim 1, wherein the second compression slot of each of the first and second fixation plates receives the second pin.

7. The method of claim 1, wherein the second fixation plate receives a first pin different from the first pin of the first fixation plate.

8. The method of claim 1, further comprising applying a medial to lateral compression between the first and second fixation plates using the first clamp.

9. The method of claim 1, wherein the securing further comprises coupling the first and second fixation plates to the first clamp, wherein the first clamp has a plurality of engagement protrusions adapted to receive corresponding engagement openings disposed on the first and second fixation plates, wherein the plurality of engagement protrusions and corresponding engagement openings align the first and second fixation plates relative to each other.

10. The method of claim 1, wherein the using a first clamp to apply a first anteroposterior compression to the first and second pins associated with the first fixation plate further comprises engaging the first and second pins with corresponding receptacles disposed on the clamping ends of the first clamp.

11. The method of claim 1, wherein the first fixation plate is configured to receive a screw at varying axes while maintaining a substantially flush engagement between an outer surface of the plate and a screw head.

12. The method of claim 1, wherein the first clamp is further configured with a drill bushing to facilitate drilling through the bones.

13. A system for applying anteroposterior compression to tarsal or carpal bones, the system comprising:
first and second fixation plates, wherein each plate has a respective anchoring hole configured to receive an anchoring bolt, wherein the anchoring bolt is configured to secure the first and second fixation plates relative to each other and the bones, and wherein the first and second fixation plates each have a first and second compression slot configured to receive a respective first and second pin; and
first and second clamps, configured to apply a first and second anteroposterior compression, respectively, to the bones using the first and second pins after the pins have been inserted through the slots and the bones.

14. The system of claim 13, wherein magnitudes of the first and second anteroposterior compressions are different.

15. The system of claim 14, wherein the first and second anteroposterior compressions are configured to create an abduction.

16. The system of claim 14, wherein the first and second anteroposterior compressions are configured to create an adduction.

17. The system of claim 13, wherein the first and second fixation plates each have a plurality of openings for coupling the plates to the first clamp.

18. The system of claim 13, wherein clamping ends of the first and second clamps have receptacles that are configured to engage the first and second pins.

19. The system of claim 13, wherein the first fixation plate is configured to receive a screws at a varying axis while maintaining a substantially flush engagement between an outer surface of the plate and a screw head.

20. The system of claim 13, wherein the first fixation plate has an amendable portion for contouring to the bones.

21. The system of claim 13, wherein the first fixation plate has a cleat to engage the bones.

22. The system of claim 13, wherein the first clamp has a drill bushing to facilitate drilling through the bones.

23. The system of claim 13, further comprising the first and second pins configured to be inserted through the bones.

24. The system of claim 13, further comprising the first and second pins configured to be inserted into the bones.

25. The system of claim 24, wherein the first pin of the first compression slot of the first fixation plate is different from the first pin of the first compression slot of the second fixation plate.

26. The system of claim 13, further comprising a variable axis screw configured to be inserted into the bones.
